(19) United States
(12) Patent Application Publication
(10) Pub. No.: US 2004/0111112 A1
(43) Pub. Date: Jun. 10, 2004

(54) Method and apparatus for retaining embolic material

(76) Inventor: Gerard von Hoffmann, Trabuco Canyon, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/717,045

(22) Filed: Nov. 19, 2003

Related U.S. Application Data
(60) Provisional application No. 60/427,842, filed on Nov. 20, 2002.

(51) Int. Cl. A61M 29/00

(52) U.S. Cl. 606/200; 623/1.15

(57) ABSTRACT

Methods and devices are disclosed for retaining embolic microcoils or other embolic materials within an aneurysm, such as a distal basilar artery aneurysm. The device includes a self expandable tubular support structure for positioning within the basilar artery. The support structure holds a barrier across the opening of the aneurysm.

This application claims priority under 35 U.S.C. § 119 to U.S. Provisional Patent Application Serial No. 60/427,842, filed Nov. 20, 2002, the disclosure of which is incorporated in its entirety herein by reference.

### BACKGROUND OF THE INVENTION

1. Field of the Invention

This invention relates generally to treatment of vascular aneurysms, and more particularly, to devices for inhibiting the escape of microcoils or other embolic agents from axial aneurysms, such as distal basal artery aneurysms.

1. Description of the Related Art

Aneurysms have been traditionally treated with externally placed clips, or internally by detachable vasoocclusive balloons or an embolus generating vasoocclusive device such as one or more vasoocclusive coils. The delivery of such vasoocclusive devices can be accomplished by a variety of means, including via a catheter in which the device is pushed through the catheter by a pusher to deploy the device. The vasoocclusive devices can be produced in such a way that they will pass through the lumen of a catheter in a linear shape and take on a complex shape as originally formed after being deployed into the area of interest, such as an aneurysm. In current techniques, the vasoocclusive devices take the form of spiral wound wires that can take more complex three dimensional shapes as they are inserted into the area to be treated. By using materials that are highly flexible, or even super-elastic and relatively small in diameter, the wires can be installed in a micro-catheter in a relatively linear configuration and assume a more complex shape as it is forced from the distal end of the catheter.

As early as 1975, metal coils were successfully used to occlude the renal arteries. Gianturco, et al., Mechanical Devices for Arterial Occlusions, 124 Am. J. Roent. 428 (1975). The purpose of the coil is to encourage quick formation of a thrombus (a blood clot) around the coil. The coils are currently in use for a wide range of treatments, and are referred to variously as occlusive coils, embolization coils, or Gianturco coils. Embolization coils of appropriate size for placement within intracranial aneurysms are commercially available from Target Therapeutics, Inc. and Cook, Inc. Embolization coils made with electrolytic mechanisms for detachment from the delivery catheter are referred to as GDC's or Guglielmi Detachable Coils. The use of GDC's is illustrated, for example, in Klein, et al., Extracranial Aneurysms and Arteriovenous Fistula: Embolization with the Guglielmi Detachable Coil, 201 Radiology 489 (1996). Use of the GDC coils within the brain is illustrated, for example, in Casasco, et al., Selective Endovascular Treatment Of 71 Intracranial Aneurysms With Platinum Coils, 79 J. Neurosurgery 3 (1993).

Because Gianturco and Guglielmi coils are often used to occlude aneurysms in critical areas of the body, it is important that they remain in place where they are implanted. However, migration of the coils after placement is a common but dangerous problem encountered with these coils. Watanabe, Retrieval Of A Migrated Detachable Coil, 35 Neuro. Med. Clin. 247 (1995) reports the migration of a coil into the basilar artery from a placement in the superior cerebellar artery. Halbach, et al., Transarterial Platinum Coil Embolization Of Carotid Cavernous Fistulas, 12 AJNR 429 (1991) reports the migration of a coil from the internal carotid artery. Migration is particularly common with coils placed in wide neck aneurysms. The possible migration of coils is a danger that must be considered in every procedure, and actual migration can be life threatening complication, since embolization at an unwanted site could occlude a critical blood flow. Migration of the coil may also represent a failure of the intended therapeutic procedure.

A variety of other embolic materials have also been deployed within cranial aneurysms. These include, among other agents, adhesives and hydrogels. Adhesives that have been introduced to help heal aneurysms include cyanoacrylates, gelatin/resorcinol/formol, mussel adhesive protein and autologous fibrinogen adhesive. Fibrin gels have also been used as sealants and adhesives in surgery, and hydrogels have been used as sealants for bleeding organs, and to create tissue supports for the treatment of vascular disease by the formation of shaped articles to serve a mechanical function. Catheters have commonly been used to introduce such therapeutic agents locally at diseased occluded regions of the vasculature to promote vessel healing. Typically a polymeric paving and sealing or aneurysm filling material in the form of a monomer solution, prepolymer solution, or as a preformed or partially preformed polymeric product, is introduced into the lumen of the blood vessel and positioned at the treatment site. The polymeric material typically can incorporate additional therapeutic agents such as drugs, drug producing cells, cell regeneration factors, and progenitor cells either of the same type as the vascular tissue of the aneurysm, or histologically different to accelerate the healing process.

Hydrogels have also been used to form expanding, swelling space-fillers for treatment of vascular aneurysms in a manner similar to other types of mechanical, embolus generating vasoocclusive devices. In one such procedure, an aneurysm is treated by inserting a hydrogel material into the vessel, and then hydrating and expanding the hydrogel material until it occludes the opening to the aneurysm, sealing it from the parent vessel. Biodegradable hydrogels have also been used as controlled-release carriers for biologically active materials such as hormones, enzymes, antibiotics, antineoplastic agents, and cell suspensions.

Vasoocclusive devices and materials and their deployment systems provide valuable treatments for diseased vascular regions. However, there remain important limitations in the technology presently available, since treating an aneurysm with coils or adhesives or occluding the aneurysm with a stent may not be completely effective in healing the vascular damage. Furthermore, when an embolus generating vasoocclusive device or space-filling device such as a vasoocclusive coil is used to treat an aneurysm, the ability to treat the aneurysm depends upon whether the embolus generating vasoocclusive device can migrate out of the aneurysm through the neck of the aneurysm. This is a particular challenge with axial bifurcation aneurysms, such as distal basilar aneurysms.

It would therefore be desirable to provide a method for sealing off the neck of an axial bifurcation aneurysm either in addition to or as an alternative to the introduction of a vasoocclusive device in the aneurysm, in order to minimize the risk of migration of an embolus generating material or device out of the aneurysm.

### SUMMARY OF THE INVENTION

There is provided in accordance with one aspect of the present invention, a basilar aneurysm occlusion device. The device comprises a radially expandable support structure, moveable between a reduced cross section for transluminal navigation and an enlarged cross section for retention within the basilar artery. At least one axially extending link extends from the radially expandable support. A basilar aneurysm patch is attached to the link, and moveable from a reduced cross section for transluminal navigation to an implanted orientation. The patch resides in an axial orientation when in the reduced cross section configuration and a transverse orientation when in the implanted orientation.

In one implementation, the support structure comprises a self expandable wire frame. The patch may additionally comprise a wire frame, and may include a membrane such as ePTFE.

There is provided in accordance with another aspect of the present invention, a method of treating a distal basilar aneurysm. The method comprises the steps of positioning an embolic material in a distal basilar aneurysm, and positioning a tubular support structure within the basilar artery such that a retention element carried by the support inhibits escape of material from the aneurysm. The positioning an embolic material step may be accomplished before, during or after the positioning a tubular support structure step. The positioning an embolic material step may comprise introducing at least one embolic microcoil into the aneurysm.

There is provided in accordance with another aspect of the present invention, a self expandable bifurcation aneurysm occlusion device. The device comprises a tubular support structure having a proximal end, a distal end and a longitudinal axis. At least one strut extends distally from the support structure, and a barrier is carried by the strut. The barrier may comprise a wire mesh, and may additionally comprise a polymeric membrane. The barrier in an unconstrained expansion resides in a plane which is transverse to the longitudinal axis. The membrane may be sufficiently porous to permit neointimal ingrowth. Alternatively, the membrane may inhibit neointimal ingrowth.

In accordance with a further aspect of the present invention, there is provided a device for obstructing the opening to an aneurysm. The device comprises a self expandable wire support, having a proximal end, a distal end and a tubular wall extending therebetween. The wall comprises a plurality of struts connected by bends. An axially oriented opening is provided at the proximal end of the support, and a transverse barrier is carried by the distal end of the support. At least one lateral opening is provided proximal to the transverse barrier, so that blood flow from the main vessel may enter the axially oriented opening and exit the lateral opening into a branch vessel. In one embodiment, the barrier is spaced distally apart from the distal end of the tubular wall. At least one, and generally two or three or four or more axially extending links join the tubular body and the barrier.

In accordance with a further aspect of the present invention, there is provided a vascular flow deflector, for implantation at a bifurcation in a vascular structure. The flow deflector comprises a support structure for positioning in a main vessel proximal to the bifurcation, the support structure having a proximal end, a distal end, and a longitudinal axis. A flow deflection surface is carried by the support structure, the flow deflection surface extending transversely across the longitudinal axis. In one implementation, the flow deflection surface comprises a surface of a wire mesh. Alternatively, the flow deflection surface comprises a surface on a polymeric membrane.

In accordance with another aspect of the present invention, there is provided a method of isolating an aneurysm. The method comprises the steps of positioning a neointimal cell growth support across the opening of an aneurysm. The support is held in position using a retention structure positioned in a vessel outside of the aneurysm. The retention structure has a longitudinal axis, and the cell growth support is positioned at an angle of at least about 45° from the longitudinal axis. Preferably, the cell growth support is positioned at an angle within the range of from about 75° to about 105° from the longitudinal axis.

In accordance with a further aspect of the present invention, there is provided an embolic coil for treating an aneurysm. The embolic coil comprises at least one embolic microcoil, and a support for retaining the microcoil in an aneurysm. A strut connects the microcoil to the support.

The support may comprise a self expandable wire structure, having a longitudinal axis. The microcoil is held by the support in a position which intersects an extension of the longitudinal axis. The support and strut may be an integral component. Alternatively, the support and the strut may be distinct components.

Further features and advantages of the present invention will become apparent to those of skill in the art in view of the detailed description of preferred embodiments which follows, when considered together with the attached drawings and claims.

### BRIEF DESCRIPTION OF THE DRAWINGS

FIG. 1 is a schematic side elevational view of an implant in accordance with the present invention.

FIG. 2 is a side elevational schematic view of an alternate implant in accordance with the present invention.

FIG. 3 is a side perspective view of a further implant in accordance with the present invention.

FIG. 4 is a detail view of a barrier design in accordance with the present invention.

FIG. 5 is a side elevational schematic view of an alternate implant in accordance with the present invention.

FIG. 6 is a side elevational cross section through a distal end of a deployment catheter in accordance with the present invention.

FIG. 7 illustrates the normal cerebral vasculature in the vicinity of the circle of Willis, and shows a deployment catheter in accordance with the present invention positioned across the basilar artery and at the opening to a distal basilar aneurysm.

FIG. 8 is an illustration as in FIG. 7, with the aneurysm barrier deployed from the deployment catheter.

FIG. 9 is an illustration as in FIG. 8, with the aneurysm barrier distally advanced to seat against the distal vessel wall.

FIG. 10 is an illustration as in FIG. 9, with the outer sheath partially retracted to partially deploy the support structure within the basilar artery.

FIG. 11 is an illustration as in FIG. 10, with the outer sheath fully retracted, to deploy the support structure within the basilar artery.

FIG. 12 is an illustration as in FIG. 11, with the deployment catheter removed from the patient.

### DETAILED DESCRIPTION OF THE PREFERRED EMBODIMENT

Referring to FIG. 1, there is illustrated a schematic view of an implant 10 in accordance with one aspect of the present invention. In general, implant 10 is dimensioned to reside within a vessel, such as an artery. In one application, the implant 10 is particularly suited to reside in the basilar artery, to treat a distal basilar aneurysm.

Implant 10 comprises a proximal end 12 and a distal end 14. A support 16 carries a barrier 18, such as through the use at least a first strut 20 and, in the illustrated embodiment, at least a second strut 22.

The support 16 comprises a wire cage 24 having a plurality of struts 26 extending in a zig-zag fashion between a plurality of proximal apexes 28 and distal apexes 30. The wire cage 24 forms a self expandable tubular structure having a central lumen 32. Self expandable zig-zag structures of this type are well known in the medical arts, such as in the context of vascular stents and grafts.

In general, the wire cage 24 is compressible to a first, low crossing profile for transluminal navigation to a treatment site, to a second, enlarged configuration for deployment at the site.

As such, any of a wide variety of cardiovascular stents can be utilized as the support 16 for the present invention. Although balloon expandable supports 16 may be utilized, a self expandable structure for support 16 is presently preferred.

A variety of self expandable structures which proof to have inadequate radial force to function as a cardiovascular stent may nonetheless be useful in the context of the present invention. This is due to the fact that the support 16 in the context of the present invention functions primarily to maintain an axial alignment of the central lumen 32 with the healthy wall of the basilar artery. This allows the struts 20 and 22 or other connectors to maintain the barrier 18 across the opening to the basilar aneurysm. When implanted, as discussed in further detail below, the barrier 18 resides against the vessel wall surrounding the basilar aneurysm. The first and second struts 20 and 22 support the wire cage 24 against downstream migration. As a consequence of the present intended use environment, the radial force generated by wire cage 24 may be less than that required by a self expanding stent intended for use to treat a vascular stenosis.

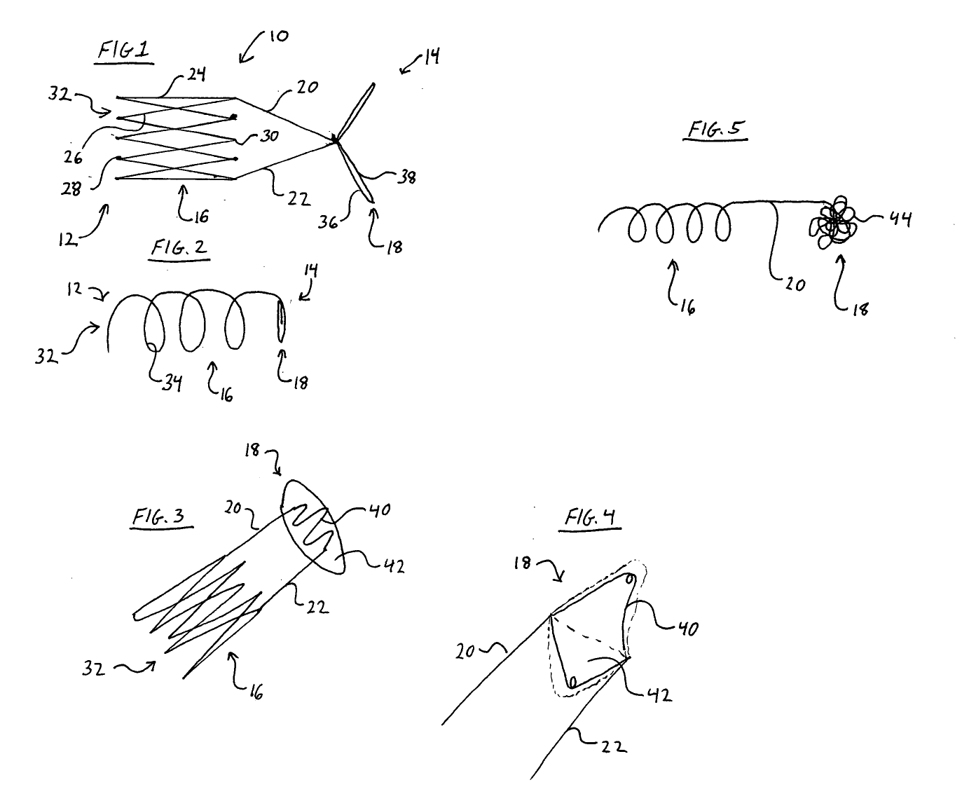

The wire cage 24 may take any of a variety of forms, as will be apparent to those of skill in the art in view of the disclosure herein. For example, referring to FIG. 2, the wire cage 24 takes the form of a spiral or pigtail 34. The wire spiral 34 defines a central lumen 32, and supports a barrier 18 by way of a strut 20. The strut 20 may be integrally formed with the wire spiral 34, as illustrated in FIG. 2. The support 16 illustrated in FIG. 2 may be deployable from a lower crossing profile catheter compared to the support 16 illustrated in FIG. 1. This may be accomplished by providing a wire with a spiral bias, and stretching the wire out linearly to fit within the deployment lumen of deployment catheter. As the wire 34 is advanced distally from the deployment lumen, it assumes the spiral configuration illustrated in FIG. 2. The cross section of the wire spiral 34, when in the reduced crossing profile configuration, is thus equal to the diameter of the wire from which it is constructed. In contrast, the minimum diameter of the support 16 in FIG. 1, in a zig-zag wire cage having, for example, three distal apexes, is at least 6 times the cross sectional area of a single wire strut.

The support 16 may be constructed from any of a variety of materials, as will be apparent to those of skill in the art. For example, metal wires such as stainless steel, nitinol, or known materials may be used. Particularly in the case of the wire spiral 34, polymeric filaments may also be utilized, in which a preset is established to bias the filament into the pigtail or spiral configuration. Polypropylene or other polymeric materials may be utilized, taking into account the thromogenisity and other properties.

The wire may be provided with any of a variety of coatings, such as to improve the thromogenisity, to encourage incorporation into the vascular intima, or to inhibit a proliferative response to injury caused by the implantation of the implant 10. Such coatings are well known in the cardiovascular stent arts, and need not be described further herein.

In addition, the wire cage 24 may be provided with a tubular sleeve such as ePTFE or Dacron. The ePTFE sleeve may have a fibril length which is selected to either encourage or inhibit a neointimal ingrowth layer.

In general, the barrier 18 comprises a proximal surface 36 and a distal surface 38. In certain embodiments, the proximal surface 36 serves as a deflection surface, to assist in deflecting the force of distal blood flow away from the distal axial aneurysm and in the direction of the branch vessel. The distal surface 38 faces the aneurysm, and may serve to retain an embolic material within the aneurysm. Thus, the implant 10 in accordance with the present invention is intended to be utilized either by itself, or in combination with any of a variety of embolic agents, such as metallic microcoils or other embolic media, some of which are described below. The nature of the barrier 18 may be modified accordingly, depending upon the structure necessary to provide adequate retention taking into account the particular embolic material for a given application.

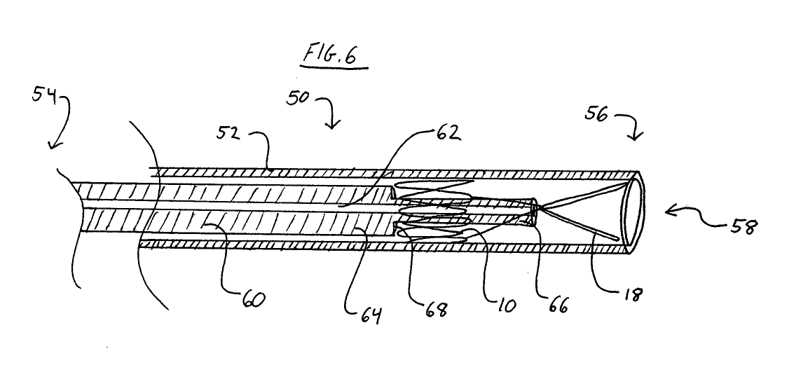

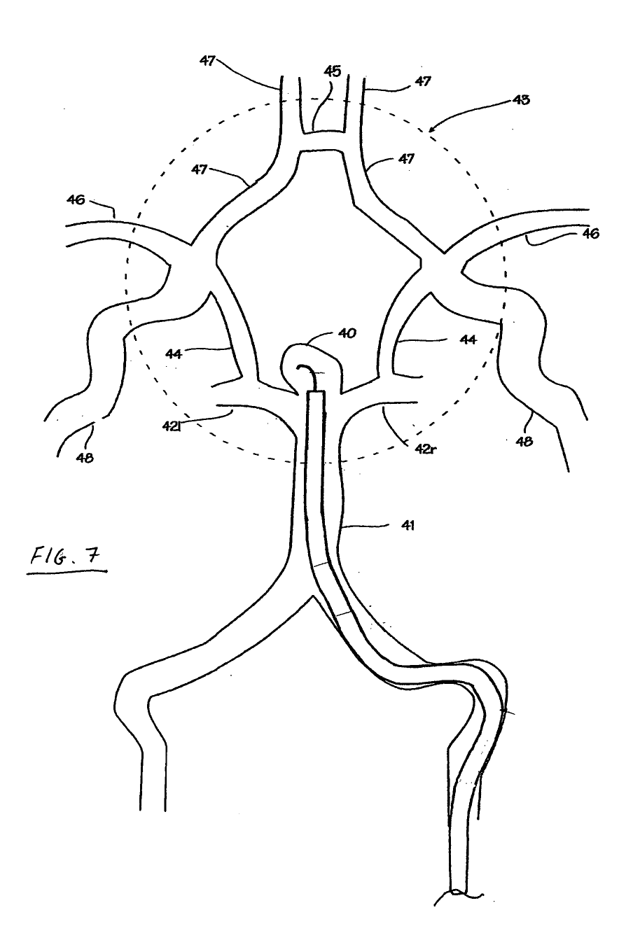

For example, referring to FIGS. 3 and 4, the barrier comprises a wire frame 40 which carries a membrane 42. The wire frame 40 may be configured in any of a variety of ways, to allow expansion from a reduced crossing profile for transluminal navigation within the deployment catheter, to an expanded cross sectional area for occluding all or a portion of the opening to the distal aneurysm.

The membrane 42 may be attached to the wire frame using any of a variety of know techniques, such as adhesive, or by embedding the wire frame within the membrane 42 or between two or more adjacent layers of the membrane 42. In accordance with one embodiment, the membrane 42 comprises a patch of ePTFE. The membrane may be attached by coating the wire frame with FEP, and thereafter thermally bonding the ePTFE membrane to the FEP coated wire frame 40.

The implant 10 is preferably provided with a membrane 42 if the implant is to be utilized primarily as a flow deflector, without filling the aneurysm with an embolic material. In addition, the use of a membrane 42 may also be desirable when the implant 10 is utilized to retain a flowable embolic material within the aneurysm. Alternatively, the membrane 42 may be omitted and the wire frame 40 may be sufficient as an embolic retention device when the implant 10 is utilized in conjunction with one or more embolic microcoils.

In accordance with a further implementation of the present invention, the barrier 18 comprises one or more microcoils 44. In current practice, a plurality of microcoils 44 are deployed from the distal end of a microcoil deployment catheter. When a sufficient length of wire or number of coils have been positioned within the aneurysm, the microcoil is detached from the catheter such as by melting a polymeric length or applying an electrical current to sever a wire. In accordance with the present invention, the microcoil 44 is integrally connected by way of a link 20 to a proximal support 16 for positioning within an artery adjacent the aneurysm. The proximal support 16 may comprise a proximal continuation of the same wire utilized to form the microcoil 44. Alternatively, the distal end of the strut 20 may be attached to the microcoil 44 in situ, through the use of a mechanical interlink, adhesives, heat bonding, or other technique.

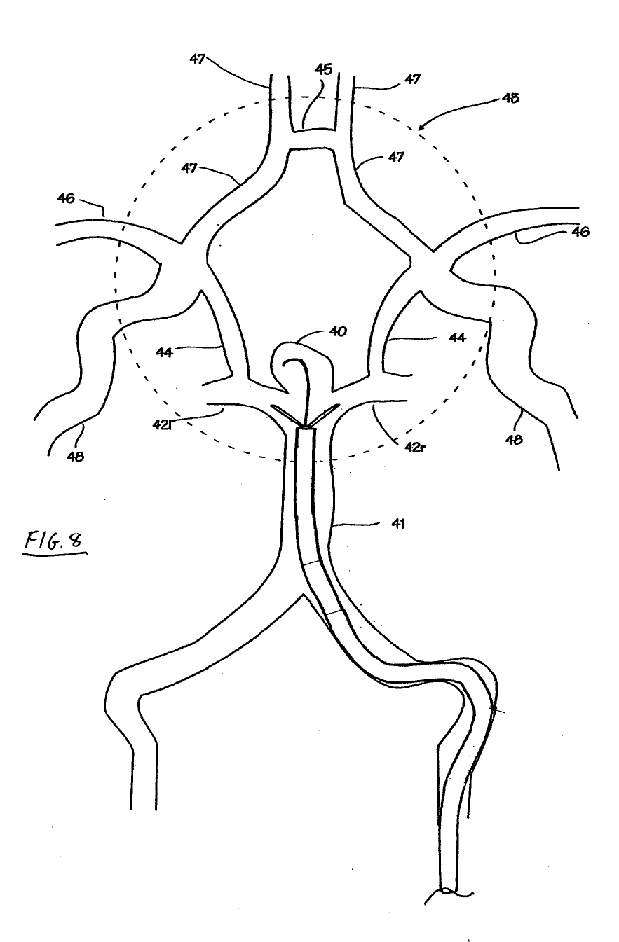

The implant 10 in accordance with the present invention may be deployed using any of a variety of deployment catheters as will be understood in the art. For example, the implant 10 according to FIGS. 2 and 5 may be deployed by advancing a prebiased wire distally from the deployment lumen in a single lumen catheter. These embodiments may provide the lowest crossing profile among the various structures disclosed herein. Alternatively, the embodiments of FIGS. 1, 3 and 4 may be deployed using a catheter such as that illustrated in FIG. 6.

Referring to FIG. 6, a distal portion of a catheter 50 is schematically illustrated. The catheter 50 comprises an elongate flexible tubular body 52, extending between a proximal end 54 and a distal end 56. At least the distal end of a tubular body 52 is provided with a central lumen 58, for retaining an implant 10 therein.

In the illustrated embodiment, the catheter 50 additionally comprises an axially moveable central core 60. The moveable core 60 comprises a guidewire lumen 62, which may also be utilized to inject embolic material and/or radioopaque dye into the treatment site. Core 60 additionally comprises a proximal section 64 having a first outside diameter, and a distal section 66 having a second, smaller outside diameter. The diameter mismatch provides an annular shoulder 68, against which the implant 10 may be seeded. In this manner, the outer tubular body 52 may be proximally retracted with respect to the core 60, to deploy the implant 10 at the treatment site. One or more retention structures such as a friction enhancing surface, one or more projections or annular ridges may be provided on the distal section 66. This will enable a partial deployment of the implant 10, and then retraction of the implant 10 back into the tubular body 52 in the event that the clinician determines the implant not suitable for a particular patient or a redeployment of the implant appears desirable. Catheter design details such as dimensions and materials are well within the skill in the art, and need not be disclosed in greater detail herein.

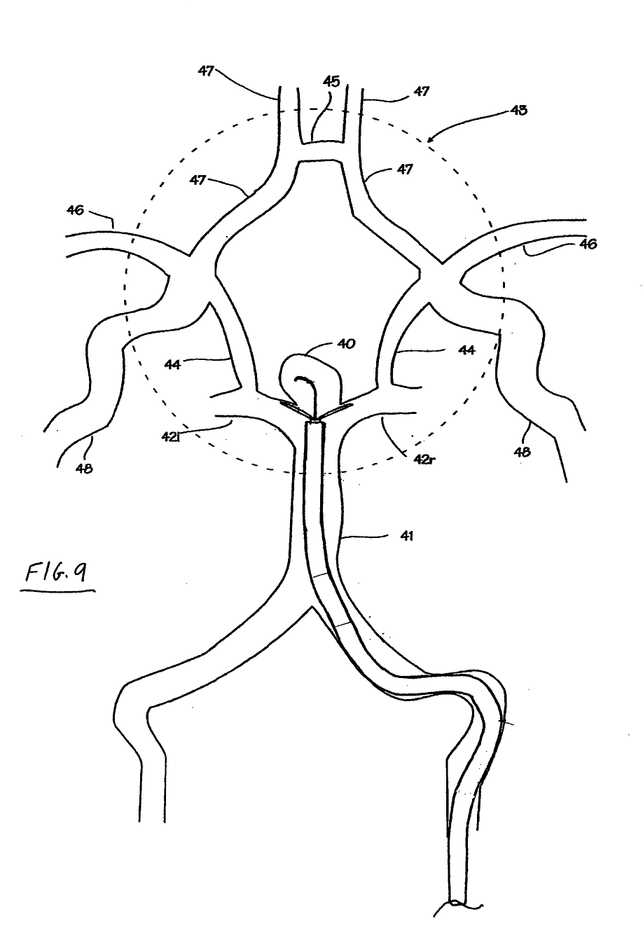

Deployment of the implant 10 will be described in connection with FIG. 7 through 12. Referring to FIG. 7, there is illustrated the normal cerebral vasculature in the vicinity of a distal basilar aneurysm. The distal end 56 of a deployment catheter 50 has been transluminally navigated into position adjacent the distal basilar aneurysm. A self expandable implant is restrained within the tubular body 52, as has been discussed.

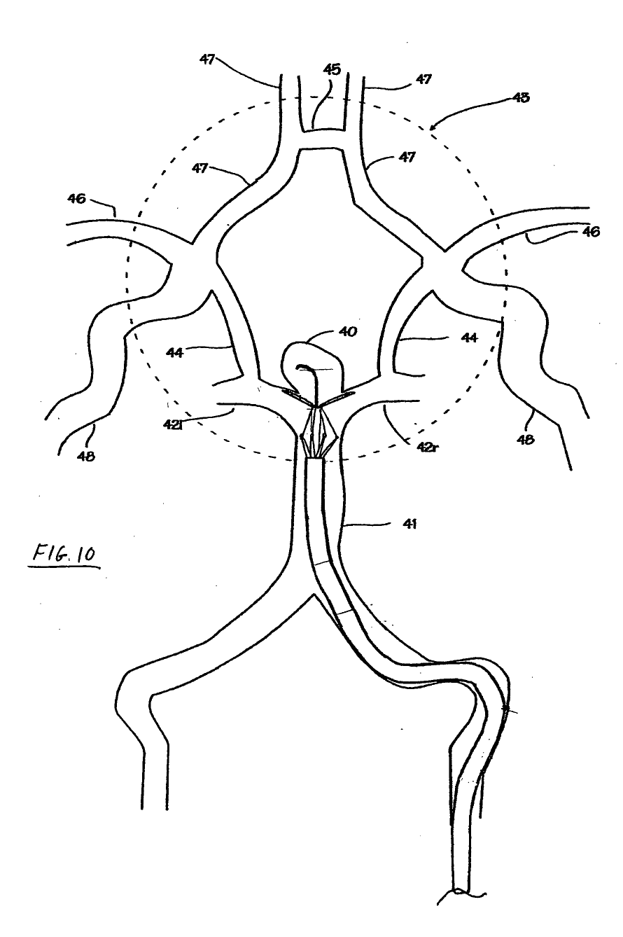

Referring to FIG. 8, the outer tubular body 52 is proximally retracted to begin deployment of the barrier 18. The illustrated implant 10 is similar to that schematically illustrated in FIG. 1, in which the barrier 18 generally comprises a butterfly like configuration.

After the barrier 18 has been released from the deployment catheter 50, the entire catheter assembly may be advanced distally as shown in FIG. 3 to seat the barrier 18 against the distal vessel wall. Since the barrier 18 inclines radially outwardly in the distal direction, it can be proximately retracted back into the deployment catheter and redeployed or removed from the patient at this point.

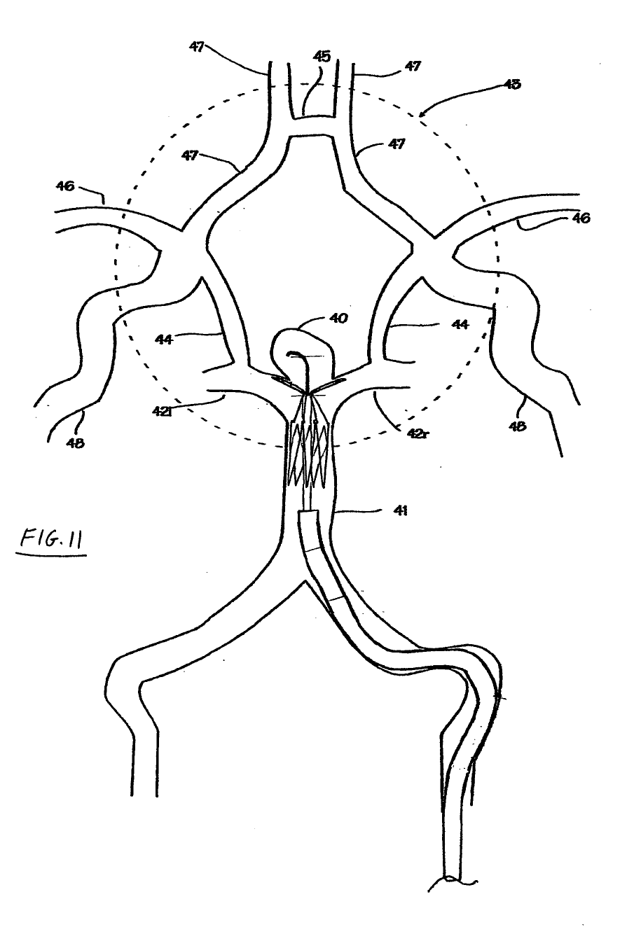

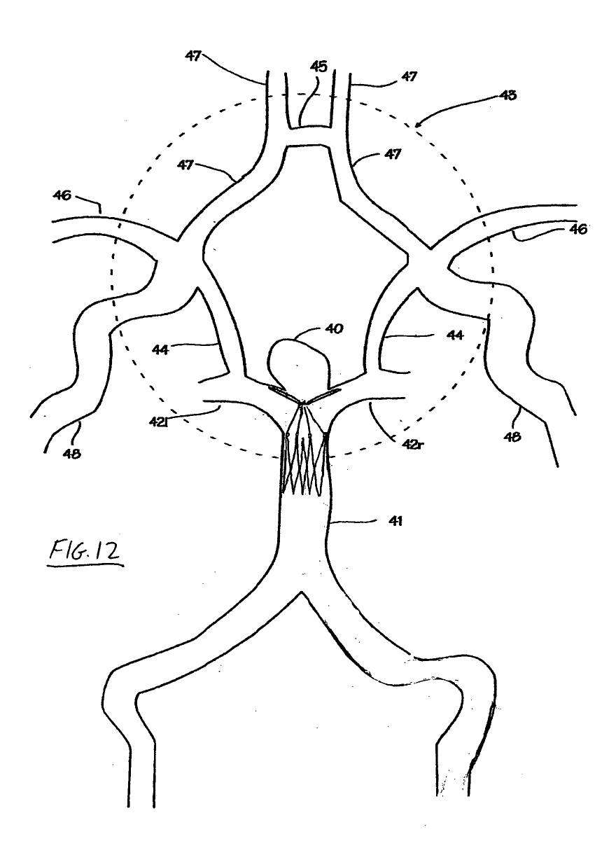

After the barrier 18 has been properly seated against the distal vessel wall, the outer tubular sleeve is proximally retracted by a second distance to begin deployment of the self expandable support 16. See FIG. 10. The position of the implant may be confirmed by injection of radioopaque dye, and the outer tubular sleeve may then be fully retracted to fully deploy the support 16. See FIG. 11. During the final deployment, the implant 10 may be retained in position against the distal vessel wall surrounding the aneurysm neck by the central core. The guidewire may or may not still be in position.

Either prior to, during or following deployment of the implant, embolic material may be introduced through the guidewire lumen into the entrapped space behind the aneurysm neck cover. The embolic material may comprise one or more microcoils such as the GDC or Microus coils, or any of a variety of polymeric embolic materials. As has been previously discussed, the nature of the barrier 18 may be varied depending upon the embolic material with which the implant 10 is intended to be used. For example, a simple transverse strut or uncovered wire structure may be sufficient to restrain embolic coils, while a structure with a smaller aperture size such as with a more dense wire mesh or weave, or a polymeric membrane, may be desirable for retaining a more flowable embolic material.

Following injection of the embolic material, the central core may be proximally retracted through the expanded support, and the catheter may be proximally retracted from the patient.

Any of a variety of conventional embolic therapies can be utilized in conjunction with the implant of the present invention. One approach is the direct injection of a liquid polymer embolic agent into the aneurysm. One type of liquid polymer used in the direct injection technique is a rapidly polymerizing liquid, such as a cyanoacrylate resin, particularly isobutyl cyanoacrylate, that is delivered to the target site as a liquid, and then is polymerized in situ. Alternatively, a liquid polymer that is precipitated at the target site from a carrier solution has been used. An example of this type of embolic agent is a cellulose acetate polymer mixed with bismuth trioxide and dissolved in dimethyl sulfoxide (DMSO). Another type is ethylene vinyl alcohol dissolved in DMSO. On contact with blood, the DMSO diffuses out, and the polymer precipitates out and rapidly hardens into an embolic mass that conforms to the shape of the aneurysm. Other examples of materials used in this “direct injection” method are disclosed in the following U.S. patents: U.S. Pat. No. 4,551,132 to Pasztor et al.; U.S. Pat. No. 4,795,741 to Leshchiner et al.; U.S. Pat. No. 5,525,334 to Ito et al.; and U.S. Pat. No. 5,580,568 to Greffet al.

Another approach that has shown promise is the use of thrombogenic microcoils. These microcoils may be made of a biocompatible metal alloy (typically platinum and tungsten) or a suitable polymer. If made of metal, the coil may be provided with Dacron fibers to increase thrombogenicity. The coil is deployed through a microcatheter to the vascular site. Examples of microcoils are disclosed in the following U.S. patents: U.S. Pat. No. 4,994,069 to Ritchart et al.; U.S. Pat. No. 5,133,731 to Butler et al.; U.S. Pat. No. 5,226,911 to Chee et al.; U.S. Pat. No. 5,312,415 to Palermo; U.S. Pat. No. 5,382,259 to Phelps et al.; U.S. Pat. No. 5,578,074 to Mirigian; U.S. Pat. No. 5,582,619 to Ken; U.S. Pat. No. 5,624,461 to Mariant; U.S. Pat. No. 5,645,558 to Horton; U.S. Pat. No. 5,658,308 to Snyder; and U.S. Pat. No. 5,718,711 to Berenstein et al.

The microcoil approach has met with some success in treating small aneurysms with narrow necks, but the coil must be tightly packed into the aneurysm to avoid shifting that can lead to recanalization. Microcoils have been less successful in the treatment of larger aneurysms, especially those with relatively wide necks. A disadvantage of microcoils is that they are not easily retrievable; if a coil migrates out of the aneurysm, a second procedure to retrieve it and move it back into place is necessary. Furthermore, complete packing of an aneurysm using microcoils can be difficult to achieve in practice. Thus, the embolic retention device of the present invention may enable the use of microcoils in axial aneurysms in the distal basilar artery.

A specific type of microcoil that has achieved a measure of success is the Guglielmi Detachable Coil (“GDC”), described in U.S. Pat. No. 5,122,136 to Guglielmi et al. Another microcoil is available from Micrus, Inc. The GDC employs a platinum wire coil fixed to a stainless steel delivery wire by a solder connection. After the coil is placed inside an aneurysm, an electrical current is applied to the delivery wire, which heats sufficiently to melt the solder junction, thereby detaching the coil from the delivery wire. The application of the current also creates a positive electrical charge on the coil, which attracts negatively-charged blood cells, platelets, and fibrinogen, thereby increasing the thrombogenicity of the coil. Several coils of different diameters and lengths can be packed into an aneurysm until the aneurysm is completely filled. The coils thus create and hold a thrombus within the aneurysm, inhibiting its displacement and its fragmentation.

The advantages of the GDC procedure are the ability to withdraw and relocate the coil if it migrates from its desired location, and the enhanced ability to promote the formation of a stable thrombus within the aneurysm. Nevertheless, as in conventional microcoil techniques, the successful use of the GDC procedure has been substantially limited to small aneurysms with narrow necks.

Still another approach to the embolization of an abnormal vascular site is the injection into the site of a biocompatible hydrogel, such as poly (2-hydroxyethyl methacrylate) (“pHEMA” or “PHEMA”); or a polyvinyl alcohol foam (“PAF”). See, e.g., Horak et al., “Hydrogels in Endovascular Embolization II. Clinical Use of Spherical Particles”, Biomaterials, Vol. 7, pp. 467-470 (November, 1986); Rao et al., “Hydrolysed Microspheres from Cross-Linked Polymethyl Methacrylate”, J. Neuroradiol., Vol. 18, pp. 61-69 (1991); Latchaw et al., “Polyvinyl Foam Embolization of Vascular and Neoplastic Lesions of the Head, Neck, and Spine”, Radiology, Vol. 131, pp. 669-679 (June, 1979). These materials are delivered as microparticles in a carrier fluid that is injected into the vascular site, a process that has proven difficult to control.

A further development has been the formulation of the hydrogel materials into a preformed implant or plug that is installed in the vascular site by means such as a microcatheter. See, e.g., U.S. Pat. No. 5,258,042 to Mehta. These types of plugs or implants are primarily designed for obstructing blood flow through a tubular vessel or the neck of an aneurysm, and they are not easily adapted for precise implantation within a sac-shaped vascular structure, such as an aneurysm, so as to fill substantially the entire volume of the structure.

U.S. Pat. No. 5,823,198 to Jones et al. discloses an expansible PVA foam plug that is delivered to the interior of an aneurysm at the end of a guidewire. The plug comprises a plurality of pellets or particles that expand into an open-celled structure upon exposure to the fluids within the aneurysm so as to embolize the aneurysm. The pellets are coated with a blood-soluble restraining agent to maintain them in a compressed state and attached to the guidewire until delivered to the aneurysm. Because there is no mechanical connection between the pellets and the guidewire (other than the relatively weak temporary bond provided by the restraining agent), however, premature release and migration of some of the pellets remains a possibility.

The embolic retention devices of the present invention can be utilized to retain any of the foregoing embolic agents within a distal axial aneurysm such as distal basilar aneurysms. The devices described herein can also be coated with any of a variety of suitable coatings, depending upon desired clinical performance. Among the coatings which could be applied are growth factors. A number of suitable growth factors include vascular endothelial growth factor (VEGF), platelet derived growth factor (PDGF), vascular permeability growth factor (VPF), basic fibroblast growth factor (bFGF), and transforming growth factor beta (TGF-beta).

Although the present invention has been described in terms of certain preferred embodiments, it may be incorporated into other embodiments by persons of skill in the art in view of the disclosure here. The scope of the invention is therefore not intended to be limited by the specific embodiments disclosed herein, but is intended to be defined by the full scope of the following claims.

What is claimed is:

1. A basilar aneurysm occlusion device, comprising:
a radially expandable support structure, moveable between a reduced cross section for transluminal navigation and an enlarged cross section for retention within the basilar artery;
at least one axially extending link; and
a basilar aneurysm patch attached to the link, and moveable between a reduced cross section orientation and an implanted orientation;
wherein the patch resides in an axial orientation when in the reduced cross section orientation and a transverse orientation when in the implanted orientation.

2. A basilar aneurysm occlusion device as in claim 1, wherein the support structure comprises a self expandable wire frame.

3. A basilar aneurysm occlusion device as in claim 2, wherein the wire frame comprises a nickel titanium alloy.

4. A basilar aneurysm occlusion device as in claim 1, wherein the patch comprises an expandable frame.

5. A basilar aneurysm occlusion device as in claim 4, wherein the patch further comprises a membrane supported by the frame.

6. A basilar aneurysm occlusion device as in claim 5, wherein the membrane comprises ePTFE.

7. A basilar aneurysm occlusion device as in claim 5, wherein the membrane supports neointimal ingrowth.

8. A method of treating a distal basilar aneurysm, comprising the steps of:
positioning an embolic material in a distal basilar aneurysm;
positioning a tubular support structure within the basilar artery such that a retention element carried by the support inhibits escape of material from the aneurysm.

9. A method of treating a distal basilar aneurysm as in claim 8, wherein the positioning an embolic material step is accomplished before the positioning a tubular support structure step.

10. A method of treating a distal basilar aneurysm as in claim 8, wherein the positioning an embolic material step is accomplished after the positioning a tubular support structure step.

11. A method of treating a distal basilar aneurysm as in claim 8, wherein the positioning an embolic material step is accomplished during the positioning a tubular support structure step.

12. A method of treating a distal basilar aneurysm as in claim 8, wherein the positioning an embolic material step comprises introducing at least one embolic coil into the aneurysm.

13. A method of treating a distal basilar aneurysm as in claim 8, wherein the positioning an embolic material step comprises introducing an embolic composition into the aneurysm.

14. A method of treating a distal basilar aneurysm as in claim 13, wherein the composition comprises a hydrogel.

15. A method of treating a distal basilar aneurysm as in claim 8, wherein the positioning a tubular support structure step comprises deploying a self expandable support into the basilar artery.

16. A method of treating a distal basilar aneurysm as in claim 8, wherein the positioning a tubular support structure step comprises deploying a balloon expandable support into the basilar artery.

17. A method of treating a distal basilar aneurysm as in claim 15, wherein the support structure comprises a wire frame.

18. A method of treating a distal basilar aneurysm as in claim 17, wherein the support structure comprises a helical coil.

19. A method of treating a distal basilar aneurysm as in claim 17, wherein the support structure comprises a zig-zag wire, having at least two longitudinal struts connected by at least one apex.

20. A method of treating a distal basilar aneurysm as in claim 8, wherein the retention element comprises at least one transverse strut for retaining at least one embolic coil within the aneurysm.

21. A method of treating a distal basilar aneurysm as in claim 20, wherein the retention element comprises a wire frame.

22. A method of treating a distal basilar aneurysm as in claim 21, further comprising a membrane on the wire frame.

23. A method of treating a distal basilar aneurysm as in claim 22, wherein the membrane is capable of supporting endothelial ingrowth.

24. A self expandable bifurcation aneurysm occlusion device, comprising:
a tubular support structure having a proximal end, a distal end, and a longitudinal axis;
at least one strut extending distally from the support structure; and
a barrier carried by the strut.

25. A self expandable bifurcation aneurysm occlusion device as in claim 24, wherein the barrier comprises a wire mesh.

26. A self expandable bifurcation aneurysm occlusion device as in claim 24, wherein the barrier comprises a polymeric membrane.

27. A self expandable bifurcation aneurysm occlusion device as in claim 24, wherein the barrier, in an unconstrained expansion, resides in a plane which is transverse to the longitudinal axis.

28. A self expandable bifurcation aneurysm occlusion device as in claim 26, wherein the membrane is porous.

29. A device for obstructing the opening to an aneurysm, comprising:
a self expandable wire support, having a proximal end, a distal end and a tubular wall extending therebetween, the wall comprising a plurality of struts connected by bends;
an axially oriented opening at the proximal end of the support;
a transverse barrier carried by the distal end of the support; and
at least one lateral opening proximal to the transverse barrier.

30. A device for obstructing the opening to an aneurysm as in claim 29, wherein the barrier is spaced distally apart from the distal end of the tubular wall.

31. A device for obstructing the opening to an aneurysm as in claim 30, further comprising at least one link extending between the distal end of the tubular body and the barrier.

32. A device for obstructing the opening to an aneurysm as in claim 31, comprising at least two axially extending links between the tubular body and the barrier.

33. A flow deflector, for implantation at a bifurcation in a vascular structure, comprising a support structure for positioning in a main vessel proximal to the bifurcation, the support structure having a proximal end, a distal end, and a longitudinal axis, and a flow deflection surface carried by the support structure, the flow deflection surface extending transversely across the longitudinal axis.

34. A flow deflector as in claim 33, wherein the flow deflection surface is a surface of a wire mesh.

35. A flow deflector as in claim 33, wherein the flow deflection surface is a surface of a polymeric membrane.

36. A method of isolating an aneurysm, comprising the steps of:
positioning a neointimal cell growth support across the opening of an aneurysm; and
holding the support in position using a retention structure positioned in a vessel outside of the aneurysm.

37. A method of isolating an aneurysm as in claim 36, wherein the retention structure has a longitudinal axis, and the cell growth support is positioned at an angle of at least about 45 degrees from the longitudinal axis.

38. A method of isolating an aneurysm as in claim 37, wherein the cell growth support is positioned at an angle within the range of from about 75 degrees to about 105 degrees from the longitudinal axis.

39. A method of isolating an aneurysm as in claim 37, wherein the holding step comprises deploying a self expandable tubular support structure in a vessel near the aneurysm.

40. An embolic coil for treating an aneurysm, comprising:
at least one embolic microcoil,
a support, for retaining the microcoil in an aneurysm; and
a strut, connecting the microcoil to the support.

41. An embolic coil as in claim 40, wherein the support is integrally formed with the microcoil.

42. An embolic coil as in claim 40, wherein the support is in contact with the microcoil.

43. An embolic coil as in claim 40, wherein the support comprises a self expandable wire structure.

44. An embolic coil as in claim 43, wherein the self expandable wire structure has a longitudinal axis, and the microcoil is held by the support in a position which intersects the longitudinal axis.

45. An embolic coil as in claim 40, wherein the strut comprises an extension of the support.
